(19) United States
(12) Patent Application Publication
(10) Pub. No.: US 2015/0141891 A1
(43) Pub. Date: May 21, 2015

(54) BODY SUPPORT CAPABLE OF SENSING TENSION APPLIED THERETO

(71) Applicant: Meng-Chun WANG, TAICHUNG HSIEN (TW)

(72) Inventor: Meng-Chun WANG, TAICHUNG HSIEN (TW)

(21) Appl. No.: 14/547,691

(22) Filed: Nov. 19, 2014

(30) Foreign Application Priority Data
Nov. 19, 2013 (TW) 102142122

(51) Int. Cl. A61F 5/02

(52) U.S. Cl. CPC A61F 5/028
USPC 602/19

(57) ABSTRACT

A body support includes a binder and a fixing segment. The binder has a left band, a right band, and an adjusting strip between the left and right bands. The left band includes a first fastener. The right band includes a second and a third fastener. When the second fastener is attached to the first fastener, the binder is positioned around a wearer's waist. The fixing segment includes a fourth fastener at its front end attached to the third fastener of the binder, and has its rear end deposited on the adjusting strip of the binder for lengthening the adjusting strip so as to adjust the level of constriction generated by the binder. The fixing segment is equipped with a tension sensor, so a care giver helping a wearer with the body support can properly adjust the tension applied to the fixing segment, thereby ensuring the wearer's wearing comfort.

### BACKGROUND OF THE INVENTION

1. Technical Field

The present invention relates to body supports for medical purposes, and more particularly to a body support capable of sensing tension applied thereto.

2. Description of Related Art

There are many types of body supports for medical purposes. Taking a waist support for example, the waist support has its elastic band mounted around a wearer's waist and fixed thereto by binding a male fastener and a female fastener at two ends of the elastic band together, so as to provide support and protection to the wearer's waist. For adjusting the level of constriction, after the male and female fasteners are separated, the elastic band is lengthened to a desired length, and then the male and female fasteners are recombined to fix the elastic band at the adjusted position where the desired level of constriction is provided.

For some physically disabled wearers (such as the elders or patients), wearing or taking off such a waist support can be only achieved under a care giver's assistance. In the event that the disabled wearer has difficulty in verbal expression, the care giver can only lengthen the elastic band according to his/her own perception without the wearer's input. In such case, it is very often the care giver over-tensions the elastic band, causing the wearer's waist constricted excessively and making the wearer uncomfortable or even injured.

### SUMMARY OF THE INVENTION

The primary objective of the present invention is to provide a body support capable of sensing tension applied thereto. Since the body support is equipped to sense a lengthening force exerted thereto by a care giver and inform the care giver of the lengthening force, the care giver can adjust the lengthening force accordingly, thereby ensuring a wearer's comfort.

For accomplishing the foregoing objective, the disclosed body support comprises a binder, a fixing segment, and a tension sensor. The binder has a left band, a right band, and an adjusting strip connected between the left and right bands. The left band has an outer side of its front end provided with a first fastener. The right band has inner and outer sides of its front end provided with a second fastener and a third fastener, respectively. The second fastener is detachably fastened to the first fastener, so as to securely mount the binder around a wearer's waist. The fixing segment has its front end provided with a fourth fastener that is configured to be detachably attached to the third fastener of the binder. The fixing segment has its rear end fixed to the adjusting strip of the binder. The tension sensor is deposited on the fixing segment, for sensing tension applied to the fixing segment.

With the aforementioned configuration, when lengthening the fixing segment to adjust the level of constriction generated by the binder through the adjusting strip, a care giver is informed of the tension applied to the fixing segment by the tension sensor, so as to avoid over-tensioning the binder, thereby maintaining the level of constriction appropriate for the wearer's comfortable wearing.

### BRIEF DESCRIPTION OF THE DRAWINGS

FIG. 1 is an applied view of the present invention.

FIG. 2 is a plane view of the present invention in an unfolded state taken from one side.

FIG. 3 is a plane view of the present invention in the unfolded state taken from the reverse side.

FIG. 4 is a front view of the present invention, showing left and right bands of the binder fastened together.

FIG. 5, similar to FIG. 4, shows the fixing segment at a fixed position.

### DETAILED DESCRIPTION OF THE INVENTION

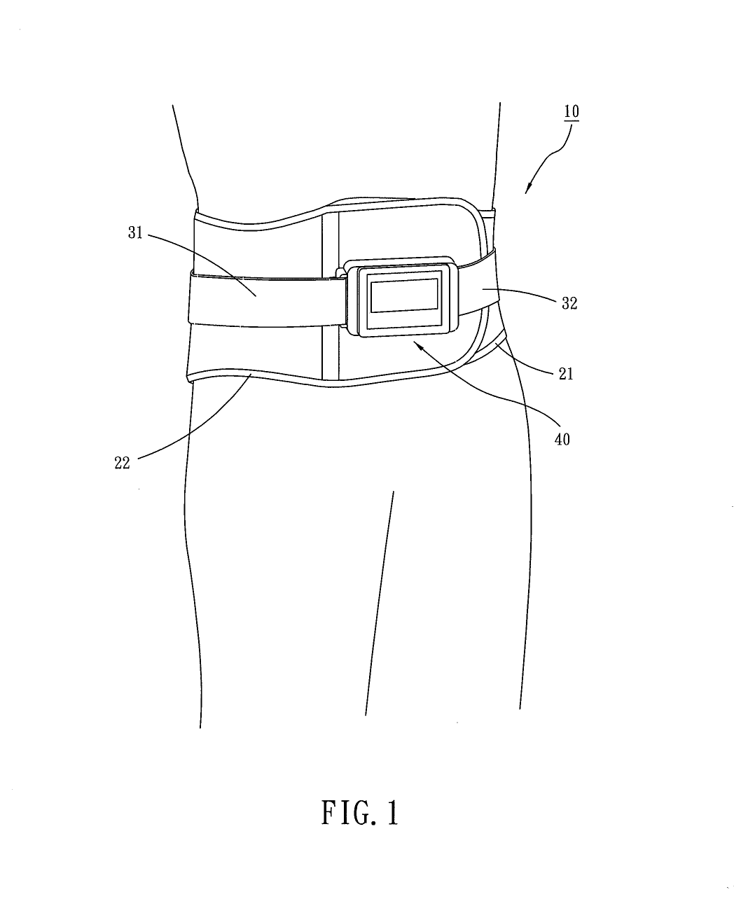

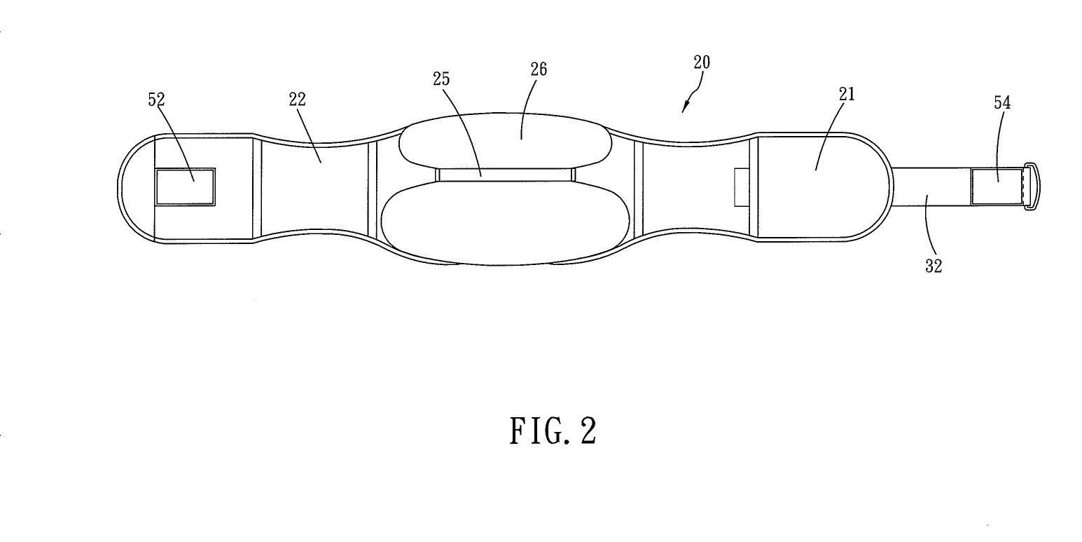

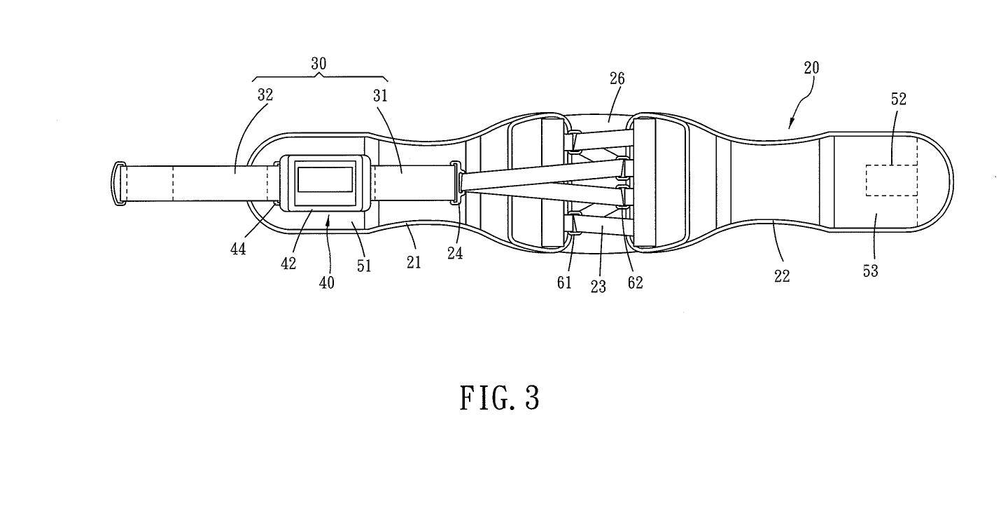

Referring to FIG. 1 through FIG. 3, according to the present invention, a body support 10, herein being a waist support, primarily comprises a binder 20, a fixing segment 30, and a tension sensor 40.

The binder 20 has a left band 21, a right band 22, and an adjusting strip 23. The left band 21 includes a first fastener 51 on the outer side of its front end. The left band 21 has its rear end provided with a plurality of first fixing buckles 61. The right band 22 has a second fastener 52 and a third fastener 53 at inner and outer sides of its front end. The right band 22 has its rear end provided with a plurality of second fixing buckles 62. One end of the adjusting strip 23 is fixed to the rear end of the right band 22 and the other end of the adjusting strip 23 passes through the first and second fixing buckles 61, 62 alternately, and additionally passes through a movable buckle 24 arranged in the halfway during this process. At last, the end of the adjusting strip 23 is also fixed to the rear end of the right band 22. Furthermore, the binder 20 has an elastic band 25 and a cushion 26. The elastic band 25 has its two ends connected to the rear end of the left band 21 and the rear end of the right band 22, respectively. The cushion 26 is attached to the elastic band 25 from inside and get positioned between the left and right bands 21, 22 by the elastic band 25, and positionally corresponding to the adjusting strip 23.

The fixing segment 30 has a first belt 31 and a second belt 32. The first belt 31 has its front end fixed to a casing 42 of the tension sensor 40, and has its rear end passing through the movable buckle 24 and then fixed to the outer side of the left band 21. The second belt 32 has its front end provided with a fourth fastener 54, and has its rear end fixed to a tab 44 of the tension sensor 40.

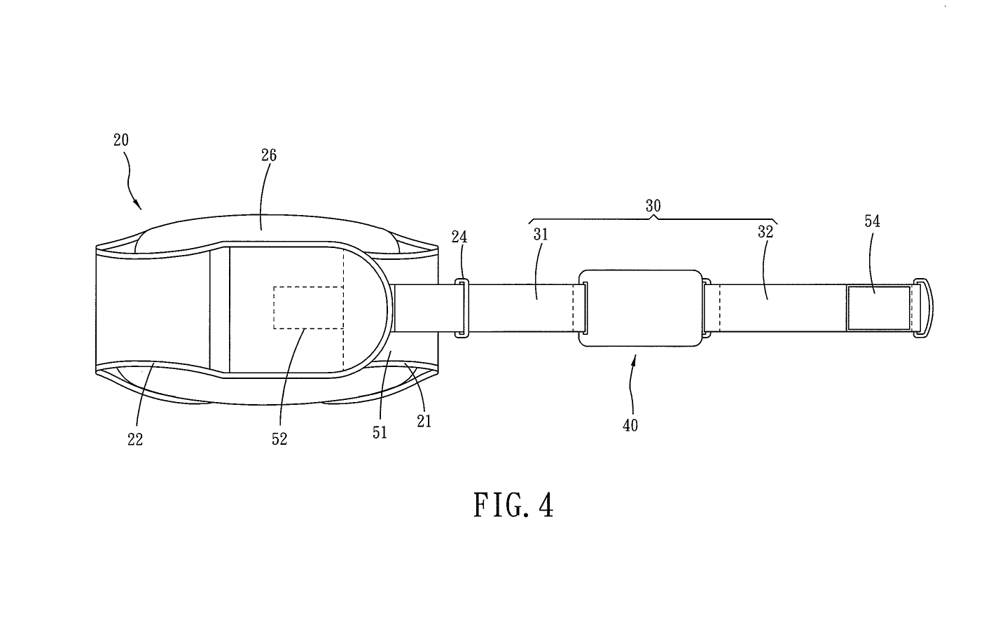

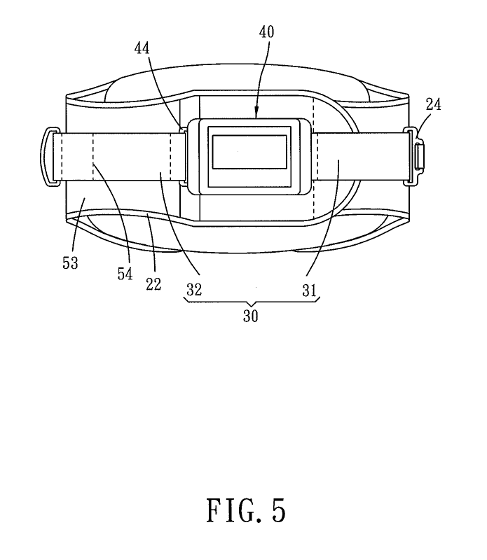

Referring to FIGS. 1, 4 and 5, when a care giver helps a wearer with the waist support, the care giver first winds the binder 20 around the wearer's waist, so that the cushion 26 is aligned with the wearer's lower back. Then the second fastener 52 of the right band 22 is attached to the first fastener 51 of the left band 21, so as to position the binder 20 around the wearer's waist. Afterward, the second belt 32 of the fixing segment 30 is lengthened, so that the second belt 32 of the fixing segment 30 draws the tab 44 of the tension sensor 40 and thereby triggers a tension sensing unit (not shown) in the tension sensor 40 to sense the tension applied to the fixing segment 30. At the same time as the second belt 32 of the fixing segment 30 is lengthened, the first belt 31 of the fixing segment 30 is affected by the tension to gradually strain the adjusting strip 23 through the movable buckle 24. As a result, the level of constriction generated by the binder 20 is increased. Thereby, the care giver can adjust the tension applied to the fixing segment 30 according to the reading given by the tension sensor 40, so as to make the level of constriction generated by the binder 20 to the wearer's waist appropriate. When the desired level of constriction of the binder 20 is achieved, the second belt 32 of the fixing segment 30 is attached to the right band 22 of the binder 20 by combining the fourth fastener 54 with the third fastener 53. The wearer now completes the wearing of the disclosed body support 10.

To sum up, the disclosed body support 10 is capable sensing the tension made by a care giver in a real-time manner, thereby allowing the care giver to adjust the binder 20 for the level of constriction that supports the wearer's comfortable wearing.

What is claimed is:

1. A body support capable of sensing tension applied thereto, comprising:
a binder, having a left band, a right band, and an adjusting strip, the left band having a front end provided with a first fastener at an outer side thereof, the right band having a front end provided with a second fastener and a third fastener at inner and other sides thereof, respectively, the second fastener being detachably attached to the first fastener, the adjusting strip being deposited between a rear end of the left band and a rear end of the right band;
a fixing segment, having a front end detachably attached to the third fastener of the binder by means of a fourth fastener, and having a rear end deposited on the adjusting strip of the binder; and
a tension sensor, being deposited on the fixing segment, for sensing the tension applied to the fixing segment.

2. The body support of claim 1, wherein the tension sensor has a casing and a tab deposited on the casing, the fixing segment having a first belt and a second belt, the first belt having a front end fixed to the casing of the tension sensor and having a rear end fixed to the outer side of the left band, the second belt having a front end provided with the fourth fastener and having a rear end fixed to the tab of the tension sensor.

3. The body support of claim 2, wherein the adjusting strip and the rear end of the first belt of the fixing segment jointly pass through a movable buckle.

4. The body support of claim 1, wherein the rear end of the left band is provided with a plurality of first fixing buckles, and the rear end of the right band is provided with a plurality of second fixing buckles, the adjusting strip alternately passing through the first and second fixing buckles and with two ends thereof fixed to the rear end of the right band.

5. The body support of claim 4, wherein between the left and right bands of the binder a cushion is provided and corresponding to the adjusting strip.

6. The body support of claim 5, wherein the binder further has an elastic band connected between the left and right bands and the cushion is attached to the elastic band.

7. The body support of claim 3, wherein the rear end of the left band is provided with a plurality of first fixing buckles, and the rear end of the right band is provided with a plurality of second fixing buckles, the adjusting strip alternately passing through the first and second fixing buckles and with two ends thereof fixed to the rear end of the right band.

8. The body support of claim 7, wherein between the left and right bands of the binder a cushion is provided and corresponding to the adjusting strip.

9. The body support of claim 8, wherein the binder further has an elastic band connected between the left and right bands and the cushion is attached to the elastic band.
